(19) United States
(12) Patent Application Publication
(10) Pub. No.: US 2015/0031581 A1
(43) Pub. Date: Jan. 29, 2015

(54) METHODS AND KITS FOR DETERMINING PROTEIN-BOUND BIOMARKERS

(71) Applicants: Rijksuniversiteit Groningen, Groningen (NL); Academisch Ziekenhuis Groningen, Groningen (NL)

(72) Inventors: Ido Peter Kema, Groningen (NL); Hermannus Johannes Roelof van Faassen, Groningen (NL); Bernhard Manz, Nordhorn (DE)

(21) Appl. No.: 14/360,798

(22) PCT Filed: Nov. 29, 2012
(86) PCT No.: PCT/NL2012/050849

§ 371 Date: May 27, 2014

Related U.S. Application Data
(60) Provisional application No. 61/567,653, filed on Dec. 7, 2011.

(30) Foreign Application Priority Data
Nov. 29, 2011 (EP) 11191169.9

(51) Int. Cl. G01N 33/538

(52) U.S. Cl. CPC G01N 33/538; G01N 2333/575; G01N 2560/00
USPC 506/12; 506/39

(57) ABSTRACT

The invention relates to the field of diagnostic methods, in particular to detection of biological molecules using mass spectroscopy (MS). Provided is an automated high-throughput method for quantitating a low molecular weight protein-bound biomarker directly in an isolated biological sample, comprising the steps of:
(a) automated prepurification of the sample using an effective amount of an acid protease under conditions that allow for digestion of one or more binding proteins; (b) applying said protease-digested sample onto an on-line SPE column to capture at least part of said biomarker, followed by sequential washing of the solid phase; (c) eluting a fraction comprising said biomarker directly onto a liquid chromatography (LC) column comprising an apolar stationary phase and subjecting it to LC-MS or LC-MS-MS measurements to determine the amount of at least one biomarker; and (d) quantitating the biomarker(s). Also provided are kits for use in such method, for instance in the determination of vitamin D derivatives, testosterone and/or or melatonin.

The invention relates to the field of diagnostic methods, in particular to methods and kits for the detection of biological molecules using mass spectrometry (MS).

Liquid chromatography(-tandem) mass spectrometry (LC-(MS/)MS) is rapidly gaining ground in special clinical chemistry laboratories. It significantly increases the analytic potential in clinical chemistry, especially in the field of low molecular weight biomarker analysis. Major shortcomings of conventional chromatographic techniques, such as labor-intensive sample preparation, long analysis times and often the relatively low specificity, are circumvented by using LC-MS/MS. In addition, LC-MS/MS has broad analyte compatibility and high analytical performance. In the last 5 years introduction of LC-MS/MS in routine diagnostics has resulted in improved assays for diagnosis and follow-up of e.g. neuroendocrine tumors characterized by the secretion of biogenic amines.

Although MS enhances the measurement of biomarkers, currently available sample preparation remains a time consuming and error prone procedure which cannot be fully automated for use in high throughput methods. Especially biomarkers that are selectively bound to binding proteins (e.g. 25 OH vitamin D and testosterone) require a release procedure, usually by disruption of the bonding by organic solvents. This procedure typically requires protein denaturation and precipitation and thereby requires (off-line) centrifugation for subsequent sample preparation. For example, WO2010/019566 discloses methods for determining the vitamin D derivatives DHVD2 and/or DHVD3 in a sample. The methods can employ LC-MS/MS techniques coupled with sample affinity purification and analyte derivatization steps. It is taught in WO2010/019566 that a deproteinized plasma sample can be extracted using an extraction column, followed by elution onto an analytical chromatography column.

WO2006/107992 also relates to off-line methods for detecting vitamin D metabolites by mass spectrometry. Whereas the method does not require analyte derivatization prior to MS analysis, samples must still be processed or purified to obtain preparations that are suitable for analysis. As in WO2010/019566, it is taught that protein precipitation is one preferred method of preparing a liquid biological sample for chromatography, e.g. for removing most of the protein from a sample leaving vitamin D metabolites soluble in the supernatant.

Van den Ouweland et al. (J. of Chromatography B 878 (2010) 1163-1168 relates to measurement of 25-OH-vitamin D in human serum using LC-MS/MS. The method comprises the release of vitamin D metabolites from binding protein by adding NaOH prior to protein precipitation.

U.S. Pat. No. 6,977,143 provides methods for determining the presence or amount of testosterone in a test sample, comprising ionizing all or a portion of the testosterone present in the sample to produce one or more testosterone ions that are detectable in a mass spectrometer. To that end, human serum is first de-proteinated using a 10% formic acid solution or a 1% trichloroacetic acid solution (in methanol). The deproteination also acts to release testosterone from SHBG, albumin, and other binding proteins. In other embodiments proteins are removed from the blood with other acids, such as a solution of 1% trichloroacetic acid in methanol.

A goal of the present inventions is the provision of an improved diagnostic method for a sample comprising at least one protein-bound analyte, which allows not only for a more specific analyte selection, but also for simplification and automation of the sample clean-up procedure. In particular, the present inventors aimed at developing an automated high throughput method for the quantification of one or more carrier-bound low molecular weight biomarkers having increased sensitivity and reduced hands-on time and handling costs. In particular, there is a need for a multi-analyte detection method allowing for the simultaneous high throughput quantification of distinct classes of biomarkers, one of which is protein bound. Preferably, the method does not involve a precipitation/centrifugation step, and does not require analyte derivatization. More preferably, it can be applied to robotic preparation of small (e.g. up to 50 μl) sample volumes.

It was found that the goals could be met by a specific combination of acid protease degradation of the binding protein and in-line immuno-affinity solid phase extraction prior to mass spectrometric quantification. Analytes do not need to be derivatized or otherwise labelled, although this procedure is not excluded for analytes that require extra analytical sensitivity. This combination of features translates into an automated sample clean-up process that can be directly coupled to LC-(MS/)MS, thereby facilitating reproducible and efficient handling of the ever growing number of samples to be analyzed in laboratories, be it in a research or clinical setting.

Accordingly, in one embodiment the invention relates to an automated high-throughput method for quantitating a low molecular weight protein-bound biomarker of interest directly in a biological sample, comprising the steps of:

(a) automated prepurification of an isolated biological sample using an effective amount of an acid protease having a pH optimum in the range of pH 1.0-5.0 under conditions that allow for digestion of one or more binding proteins until they can no longer bind the biomarker;
(b) applying said protease-digested sample onto an on-line SPE column comprising an immunoaffinity sorbent comprising an antibody capable of binding the at least one biomarker of interest, followed by sequential washing of the solid phase;
(c) eluting from said immunoaffinity SPE column a fraction comprising said biomarker directly onto a liquid chromatography (LC) column comprising an apolar stationary phase and subjecting the eluate to LC-MS or LC-MS-MS measurements to determine the amount of at least one biomarker; and
(d) quantitating the biomarker(s) in the sample by correlation with standard samples.
Digestion of a carrier protein in methods for detecting protein-bound analytes is known in the art.

Blasco et al. (J. Chromatogr. A 1154 (2007) 230-239 reported the analysis of meat samples for anabolic steroid residues by LC/MS-MS. The sample preparation method involves enzymatic digestion followed by several rounds of extraction with methanol. The only enzyme disclosed is subtilisin, which is not an acid protease.

EP2126586 (also published as WO2008/092917) relates to the quantitative determination of vitamin D metabolites in blood serum or plasma by competitive binding analysis. It discloses sample pretreatment with a subtilisin-type serine protease (only proteinase K is exemplified) to digest vitamin D binding proteins in the sample, followed by significant sample dilution in a buffer which ensures that the protease does not disturb the competition binding analysis. This step of sample dilution is highly undesirable when low amounts of analytes are present. Furthermore, EP2126586 fails to teach the use of an acid protease, let alone in combination with in-line solid phase extraction and mass spectrometric identification and quantification.

The term “acid protease” as used herein refers to a protein-digesting enzyme that exhibits maximum activity and stability in acid conditions (pH 1.0-5.0) and is inactivated at pH values above 6.0. Acid proteases have a low isoelectric point and are low in basic amino acids. Two types are widely used in the food and beverage industries: those from Aspergillus, which resemble pepsin; and those from Mucor, which resemble rennin. Preferably, the acid protease is a member of the aspartate protease family. Aspartic proteases are a family of protease enzymes that use an aspartate residue for catalysis of their peptide substrates. In general, they have two highly-conserved aspartates in the active site and are optimally active at acidic pH. Nearly all known aspartyl proteases are inhibited by pepstatin. Aspartic endopeptidases (EC 3.4.23) of vertebrate, fungal and retroviral origin have been characterised. More recently, aspartic endopeptidases associated with the processing of bacterial type 4 prepilin and archaean preflagellin have been described. Eukaryotic aspartic proteases include pepsins, cathepsins, and renins.

Very good results were obtained when pepsin was used to digest carrier protein. Pepsin is most efficient in cleaving peptide bonds between hydrophobic and preferably aromatic amino acids such as phenylalanine, tryptophan, and tyrosine. Pepsin from porcine gastric mucosa (pepsin A), is widely commercially available e.g. as lyophilized powder. Pepsin treatment can be conveniently performed at room temperature e.g. for 30-90 minutes using a 0.4% solution of pepsin in 1 M hydrochloric acid.

The in-sample digestion of carrier protein to release the biomarker of interest obviates the need for laborious or error prone steps to dissociate (e.g. by acid treatment or acetonitrile precipitation) and physically separate the binding protein from the biomarker by centrifugation. Furthermore, conventional protein precipitation techniques are incompatible with in-line SPE since any trace of precipitate would severely disturb the subsequent immuno affinity chromatographic process, like clogging of the tubing, valves etc. Accordingly, the method of the invention involves the application of the protease-digested sample onto the online immunoaffinity SPE column without prior protein precipitation and/or centrifugation step.
A method of the invention is not disclosed or suggested in the art.

Shao et al. (Analytica Chim Acta 548 (2005) 41-50 describes the simultaneous determination of synthetic steroids in meat by liquid chromatography-tandem MS spectrometry. The method involves treatment with β-glucuronidase/arylsulfatase followed by precipitation by methanol and sample centrifugation, and extraction of the supernatant with n-hexane before subjecting the sample to Sep-Pak silica or amino-propyl SPE cartridges. It does not relate to acid protease treatment in combination with sample clean-up by immunoaffinity

Wong et al. (Analytica Chimica Acta 697 (2011) 48-60 discloses a broad-spectrum equine urine screening method for free and enzyme-hydrolysed conjugated drugs with ultraperformance LC/MS. The procedure may comprise sample treatment with β-glucuronidase and an unspecified protease, followed by sample dilution and centrifugation before subjecting it to SPE.

WO2006/010369 relates to methods for determining proteins and protein-bound compounds, like cobalamin and cobalamin-binding proteins, comprising enzymatic modification. Exemplary protein modifications include deglycosylation and proteolytic degradation. Preferred proteolytic enzymes include serine protease, subtilisin, aspartic protease and thermolysin. The examples of WO2006/010369 only disclose the use of chymotrypsin, proteinase K, subtilisin and thermolysin and conclude that proteinase K and subtilisin are the most effective. WO2006/010369 fails to teach or suggest the advantageous combination of sample treatment with acid protease, sample clean-up with immunoaffinity SPE and analyte quantification using LC-MS(/MS).

Weltring et al. (Physiology & Behaviour 105 (2012) 510-521 developed a method to measure steroid hormones with LC-MS/MS in faecal samples. The method involves hydrolysing of sample extracts with glucuronidase to deconjugate glucuronides, followed by extraction of free steroids by liquid-liquid extraction and purification of the aqueous layers by C-18 SPE. Additional steps prior to LC-MS/MS analysis involve further extraction steps and solvolysis using a mixture of ethylacetate/sulphuric acid.

The term “in-line” as used herein refers to steps performed without further need for operator intervention. For example, by careful selection of valves and connector plumbing, two or more chromatography columns can be connected such that material is passed from one to the next without the need for additional manual steps. In preferred embodiments, the selection of valves and plumbing is controlled by a computer pre-programmed to perform the necessary steps. Most preferably, the chromatography system is also connected in such an in-line fashion to the detector system, e.g., an MS system. Thus, an operator may place a tray of samples in an autosampler, and the remaining operations are performed “in-line” under computer control, resulting in purification and analysis of all samples selected.

In contrast, the term “off-line” as used herein refers to a procedure requiring manual intervention of an operator after the test sample is loaded onto the first column. Thus, if samples are subjected to precipitation, and the supernatants are then manually loaded into an autosampler, the precipitation and loading steps are off-line from the subsequent steps.

The MS-based identification and quantification method of the invention does not require derivatization or radiolabelling of the biomarker. However, in certain cases it may be advantageous to derivatize the biomarker, for instance if the biomarker is present in very low concentrations derivatization can enhance sensitivity of detection by improving the signal-to-noise ratio.

As described above, a method of the invention was developed to overcome the technical problems encountered with the (high throughput) detection and quantitation of protein-bound biomarkers, in particular the simultaneous detection of multiple biomarkers in a single sample, at least one of which is a low molecular weight protein-bound biomarker. As used herein, the term “low molecular weight protein-bound biomarker” refers to a biological molecule up to about 1000 g/mol which occurs in the circulation in a protein-bound form. Typically, binding is reversible and the analyte circulates both in a protein-bound and free (e.g. dialyzable non-bound) form. The skilled person will understand that the LC-MS based detection method will detect the total amount of biomarker i.e. the fraction that was initially bound to the carrier protein as well as the free form.

The biomarker of interest can be of any nature. Preferably, it is of a non-proteinaceous nature since this ensures resistance to proteolytic degradation by the acid protease. Protein-bound biomarkers are often hydrophobic. Well known protein-bound (non-proteinaceous) biomarkers include steroids, sterols and indoles.

A steroid hormone (abbreviated as sterone) is a steroid that acts as a hormone. Steroids have strong biological activity at very low concentrations in target tissues and, therefore, the analysis of steroids in body fluids or tissues is necessary to elucidate the nature of the many endocrine disease processes and thus be useful for diagnosis and treatment. Steroid hormones can be grouped into five groups by the receptors to which they bind: glucocorticoids, mineralocorticoids, androgens, estrogens, and progestogens. Vitamin D derivatives are a sixth closely related hormone system with homologous receptors, though technically they are sterols rather than steroids.

A method of the invention may also be used to quantitate synthetic analogs of naturally occurring protein-bound molecules. A variety of synthetic steroids and sterols have been contrived. Most are steroids, but some non-steroidal molecules can interact with the steroid receptors because of a similarity of shape. Some synthetic steroids are weaker, and some much stronger, than the natural steroids whose receptors they activate. Examples of synthetic steroid hormones include the glucocorticoids prednisone, dexamethasone and triamcinolone; the mineralocorticoid fludrocortisone; the Vitamin D analog dihydrotachysterol; the androgens oxandrolone, testosterone and nandrolone (also known as anabolic steroids); the oestrogens diethylstilbestrol (DES) and the progestins norethindrone and medroxyprogesterone acetate.

In one embodiment, the biomarker is a vitamin D metabolite. Bioactive vitamin D or calcitriol is a steroid hormone that has long been known for its important role in regulating body levels of calcium and phosphorus, and in mineralization of bone. More recently, it has become clear that receptors for vitamin D are present in a wide variety of cells, and that this hormone has biologic effects which extend far beyond control of mineral metabolism. The term “vitamin D” is a term referring to one or more members of a group of steroid molecules. Vitamin D3, also known as cholecalciferol, is generated in the skin of animals when light energy is absorbed by a precursor molecule 7-dehydrocholesterol. Vitamin D is thus not a true vitamin, because individuals with adequate exposure to sunlight do not require dietary supplementation. There are also dietary sources of vitamin D, including egg yolk, fish oil and a number of plants. The plant form of vitamin D is called vitamin D2 or ergosterol. However, natural diets typically do not contain adequate quantities of vitamin D, and exposure to sunlight or consumption of foodstuffs purposefully supplemented with vitamin D are necessary to prevent deficiencies. Vitamin D, as either D3 or D2, does not have significant biological activity. Rather, it must be metabolized within the body to the hormonally-active form known as 1,25-dihydroxycholecalciferol. Each of the forms of vitamin D is hydrophobic, and is transported in blood bound to carrier proteins. The major carrier is called, appropriately, vitamin D-binding protein. The half-life of 25-hydroxycholecalciferol is several weeks, while that of 1,25-dihydroxycholecalciferol is only a few hours. The vitamin D-binding protein (DBP), also called Group Specific Component (Gc) is a glycoprotein present in the plasma of most vertebrates and it has a molecular weight of about 52.000 D(alton) in humans. It is the main carrier for vitamin D and its hydroxylated metabolites in the plasma of vertebrates, showing the highest affinity for 25-hydroxy-cholecalciferol. In a preferred embodiment, the invention provides an automated high-throughput method for quantitating 25-hydroxyvitamin D3, 25-hydroxyvitamin D2, 1α,25-dihydroxyvitamin D2 and/or 1α,25-hydroxyvitamin D3 directly in an isolated biological sample. Most preferably, the biomarker is 25-hydroxyvitamine D3.

In another aspect, the biomarker is a sex hormone, such as testosterone Testosterone is a steroid hormone from the androgen group and is found in mammals, reptiles, birds, and other vertebrates. In mammals, testosterone is primarily secreted in the testes of males and the ovaries of females, although small amounts are also secreted by the adrenal glands. It is the principal male sex hormone and an anabolic steroid. The majority of circulating testosterone in men comes from production in the interstitial cells of Leydig at the testicles. The adrenal gland also produces small amounts. Regulation of testicular production occurs via a negative feedback loop system involving the anterior pituitary, hypothalamus, and testicles; referred to as the hypothalamic-pituitary-testicular axis. The majority of the circulating testosterone is transported bound to various carrier proteins (sometimes referred to as binding proteins). The principal carrier protein is sex steroid-binding globulin (SSBG), however other plasma proteins can also bind and carry testosterone to a lesser degree (e.g., albumin, cortisol-binding globulin). The remaining non-bound circulating testosterone is referred to as free testosterone. This free testosterone and, notably, its conversion product dihydrotestosterone, are considered the biologically active forms of the steroid, as these hormones can specifically interact at the target tissue receptors. Circulating bound and free testosterone are collectively referred to as total testosterone; sometimes in the literature the term testosterone is used synonymously for total testosterone. Testosterone levels are much lower in females compared to males. The major sources of testosterone in females are the ovaries, the adrenal glands, and the peripheral conversion of precursors, specifically the conversion of androstenedione to testosterone. In females, the normal levels of androgens may provide a substrate for estrogen production. Increased serum testosterone levels in females may be indicative of polycystic ovary syndrome and adrenal hyperplasia, among other conditions. The clinical manifestations of excess testosterone in females include infertility, hirsutism, amenorrhea, and obesity. Numerous assays for testosterone are known to those of skill in the art. See, e.g., Marcus and Durnford, Steroids 46: 975-86 (1985); Giraudi et at, Steroids 52: 423-4 (1988); Ooi and Donnelly, Clin. Chem. 44: 2178-82 (1988); Dorgan et al., Steroids 67: 151-8 (2002); Choi et al., Clin. Chem. 49: 322-5 (2003).

As a further example, the biomarker is an indole, defined as an aromatic bicyclic structure, consisting of a six-membered benzene ring fused to a five-membered nitrogen-containing pyrrole ring. Indole is a popular component of fragrances and the precursor to many pharmaceuticals. In one embodiment, the biomarker is a tryptophan-derived tryptamine alkaloid, like the neurotransmitters serotonin and melatonin.

Serotonin or 5-hydroxytryptamine (5-HT) is a monoamine neurotransmitter. Biochemically derived from tryptophan, serotonin is primarily found in the gastrointestinal (GI) tract, platelets, and in the central nervous system (CNS) of animals including humans. It is a well-known contributor to feelings of well-being; it is also known to contribute to happiness, although its exact role in the central nervous system needs to be delineated and therefore is the focus of many research projects. Approximately 80 percent of the human body's total serotonin is located in the enterochromaffin cells in the gut, where it is used to regulate intestinal movements. The remainder is synthesized in serotonergic neurons in the CNS where it has various functions. These include the regulation of mood, appetite, and sleep. Serotonin also has some cognitive functions, including in memory and learning. Recently, regulation of bone mass was shown to be under the control of serotonin signalling. Modulation of serotonin at synapses is thought to be a major action of several classes of pharmacological antidepressants. Accurate detection and quantification of serotonin is of particular clinical interest e.g. for monitoring serotonin metabolism in various disorders. Melatonin is a hormone secreted by the pineal gland in the brain. It helps regulate other hormones and maintains the body's circadian rhythm. The circadian rhythm is an internal 24-hour “clock” that plays a critical role in when we fall asleep and when we wake up. Approximately 60-70% of melatonin is bound reversibly to proteins. With the acid protease treatment it is possible to release bound melatonin, and total melatonin can be determined.

A method of the invention can be applied to analysis of any type of isolated biological or non-biological sample. In one embodiment, it is a biological sample e.g. a sample obtained from a mammalian subject, preferably a human subject or a test animal. The sample is for example blood, urine, lachrymal, plasma, serum or saliva. Preferably it is a blood plasma sample

In another embodiment, the biological sample is an adipose tissue sample, such as a subcutaneous or visceral fat sample. Preferably, the adipose sample is pre-treated using organic liquid-liquid extraction.

A fully automated method of the invention is characterized by a minimal sample loss and/or sample dilution. This allows for the use of very small sample volumes. For example, good results can be obtained with samples volumes as small as 50 microliter, preferably up to 35 microliter. This opens up a new window of applications, both in a clinical or in a research setting. For example, samples obtained by automated sampling of a biological fluid from a (small) laboratory animal can be readily analyzed. In one embodiment, sampling is performed over a predetermined time period with predetermined time intervals. This is particularly interesting for the analysis of biomarkers that are (suspected of) being produced in a circadian rhythm. Specialized devices are available to automatically take (blood) samples over a predetermined time period, like a 2, 8, 12, or 24 hr time period with predetermined intervals e.g. 5-30 minute intervals.

To monitor sample recovery and to correct for any possible sample loss, it is advantageous to add a labelled tracer molecule. The tracer molecule is preferably a labelled analog of the relevant biomarker of interest. A stable-isotope labelled e.g. deuterated or carbon-13 version of the biomarker of interest is preferred.

Following sample pretreatment with an acid protease, the protease-treated sample containing acid protease may be neutralized to a pH value at which the acid protease is substantially inactive, e.g. above pH 6.0. Neutralization avoids that the protease disturbs any subsequent clean-up steps, in particular antibody-mediated capture of the biomarker by immunoaffinity SPE. Neutralization is readily accomplished by the addition of an appropriate buffer. Useful neutralization reagents include sodium hydroxide, potassium hydroxide, tris(hydroxymethyl)aminomethane and sodium carbonate. Suitably, 1 M sodium hydroxide is used. To ensure optimal detection sensitivity, it is important that the sample is not significantly diluted during neutralization. Preferably, the neutralization does not increase the volume of the sample to more than 110%.

Step (b) of the method comprises applying the protease-digested sample onto an on-line SPE column comprising a suitable immunoaffinity sorbent to capture at least part of the biomarker on the solid phase.

The sorbent in the SPE cartridge comprises one or more types of immunoaffinity sorbent comprising an immobilized antibody capable of binding the at least one biomarker of interest. Numerous suitable sorbents are known to those skilled in the art. See, e.g. Ruhn et al., J Chrom A 669: 9-19 (1994); Brne et al., J Chrom A 1216: 2658-2663 (2009); Moser et al., Bioanalysis 2: 766-790 (2010). Capture by an immunoaffinity sorbent is based on the affinity between antibody and antigen (biomarker) caused by molecular recognition. Immunoaffinity sample clean-up is particularly useful in case of difficult sample matrices such as plasma or urine. Typically, the column material is solvated to activate the antibody. Subsequently, the sample is loaded on the column in such a way that interaction can occur. For instance, extreme pH ranges and/or salt concentrations are avoided. Thereafter, non-specific matrix components are washed away. In the elution step, the specific antibody-antigen binding is disrupted in order to release the biomarker(s) for further analysis.

Preferably, the immobilized antibody has a low avidity and high affinity for the biomarker. The term “avidity” refers to the combined strength of multiple bond interactions. Avidity is distinct from affinity, which is the strength of a single bond. As such, avidity is the combined synergistic strength of bond affinities rather than the sum of bonds. It is commonly applied to antibody interactions in which multiple antigen binding sites simultaneously interact with a target. Individually, each binding interaction may be readily broken, however, when many binding interactions are present at the same time, transient unbinding of a single site does not allow the molecule to diffuse away, and binding of that site is likely to be reinstated. The overall effect is synergistic, strong binding of antigen to antibody (e.g. IgM is said to have low affinity but high avidity because it has 10 weak binding sites as opposed to the 2 strong binding sites of IgG, IgE and IgD).

In one embodiment, the antibody as a relatively non-specific antibody that can retain a group of structurally related biomarkers of interest for subsequent MS-based identification and quantification. The non-specific antibody can be a monoclonal or polyclonal antibody raised against a common structural motif which is present in the two or more structurally related biomarkers, for example the tetracyclic carbon rings characteristic for steroids. Exemplified herein below is the capture of testosterone using a polyclonal antibody raised against the A-ring of the steroid backbone.

In a specific embodiment, the immunoaffinity sorbent comprises at least two distinct antibodies, each antibody being specific for a different biomarker of interest, wherein at least one of the biomarkers is protein-bound. The biomarkers of interest may also both be protein-bound. Thus, provided herein is an automated high-throughput method for the simultaneous quantitation of at least two low molecular weight biomarkers, at least one of which is protein-bound, directly in an isolated biological sample, comprising the steps of:

(a) automated prepurification of the isolated biological sample using an effective amount of an acid protease having a pH optimum in the range of pH 1.0-5.0 under conditions that allow for digestion of one or more binding proteins until they can no longer bind the protein-bound biomarker of interest;

(b) applying said protease-digested sample onto an on-line immunoaffinity SPE column to capture the at least two biomarkers without prior precipitation and/or centrifugation, followed by sequential washing of the solid phase; wherein the immunoaffinity SPE column comprises an immunoaffinity sorbent comprising at least two distinct antibodies, each antibody capable of binding a biomarker of interest;

(c) eluting from the SPE column a fraction comprising said at least two biomarkers directly onto a liquid chromatography (LC) column comprising an apolar stationary phase and subjecting it to LC-MS or LC-MS-MS measurements to determine the amount of each biomarker; and

(d) quantitating the biomarkers in the sample by correlation with standard samples.

The in-sample acid protease treatment and in-line immunoaffinity SPE allows for a simultaneous detection of both classes of biomarkers in a high throughput. For example, the sorbent is a mixture of a first resin conjugated to an antibody capable of binding a steroid hormone, e.g. testosterone, and a second resin conjugated to an antibody capable of binding a metanephrine. In another embodiment, the invention provides an automated “multi-analyte” method for the simultaneous quantitation of at least three or at least four distinct biomarkers. See Example 5 herein below, demonstrating the capture of a vitamin D derivative, metanephrine, normetanephrine and 3-methoxytyramine using a mixture of four different immunoaffinity sorbents in a single SPE cartridge. One antibody showing cross-reactivity may be used to capture different structurally related metabolites.

Biomarker capture is followed by sequential washing of the solid phase with a mobile phase to remove unbound sample components. The skilled person will be able to determine the optimal conditions. Suitable washing mobile phase include 10% acetonitrile in H₂O (or 10% methanol in H₂O), additives like formic acid or ammonium hydroxide can be added (suitable concentrations are 0.2%). Typically, the SPE column is washed with at least 40, preferably at least 50, more preferably at least 60 column volumes.
Step (c) of a method according to the invention comprises eluting a fraction comprising at least one biomarker, preferably a mixture of biomarkers directly onto a liquid chromatography (LC) column comprising an apolar stationary phase and subjecting it to LC-(MS/)MS to determine the amount of the at least one biomarker. Non-polar LC stationary phases are known in the art. For example, the HPLC stationary phase comprises octadecyl chains, preferably on an endcapped silica backbone (Silica-C18). In a specific aspect, the HPLC column comprises Fused-Core particles manufactured by fusing a porous silica layer onto a solid silica particle. Columns of this type can be run at higher mobile phase linear velocity and still maintain their resolving power, so that higher sample throughput can be achieved. Suitable columns include those commercially available under the tradenames: HALO (Advanced Materials Technology), Kinetex (Phenomenex), and Ascentis Express (Supelco).

In another embodiment, the LC column is a charged surface hybrid (CSH™) column developed by Waters, Inc. The SCH technology provides several advantages, including superior peak shape for basic compounds, increased loading capacity, rapid column re-equilibration after changing mobile-phase pH, improved batch-to-batch reproducibility and excellent stability at low pH. For example, an XSelect CSH fluoro-phenyl column is advantageously used for the simultaneous detection of vitamin D derivatives and metanephrines.

The SPE column and the LC column are preferably part of an XLC system wherein sample storage, automated sampling, automated on-line SPE and HPLC are integrated. Examples are Prospekt or Symbiosys systems from Spark Holland (Emmen, The Netherlands). The mobile phase leaving the LC column and comprising the biomarker(s) of interest is carried via a so-called “probe” to the ionisation chamber of the MS(-MS) detector. As used herein, the abbreviation LC-(MS/)MS refers to high performance liquid chromatography coupled to (tandem) mass spectrometric detection. Tandem MS is preferred. LC-(MS/)MS combines the physical separation capabilities of HPLC with the high analytical analytical sensitivity, specificity and accuracy of mass spectrometric detection. MS can use atmospheric pressure chemical ionization (APCI) in the positive ion mode or electrospray ionization (ESI) to generate precursor positive ions. Analytes of interest can exist as charged species, such as protonated molecular ions [M^{o}+H⁺] or [M+H⁺] in the mobile phase. During the ionization phase, the molecular ions are desorbed into the gas phase at atmospheric pressure and then focused into the mass spectrometer for analysis and detection. Additional information relating to atmospheric pressure chemical ionization is known to those of skill in the art; see U.S. Pat. No. 6,692,971.

MS analysis can be conducted with a single mass analyzer (MS) or a “tandem in space” analyzer such as a triple quadrupole tandem mass spectrometer (MS/MS). Using MS/MS, the first mass filter (Quadruple 1, Q1) can select, or can be tuned to select, independently, one or more of the molecular ions of the biomarker(s) of interest, and the internal standard. The second mass filter (Q3) is tuned to select specific product or fragment ions related to the analyte of interest. Between these two mass filtration steps, the precursor molecular ions can undergo collisionally-induced dissociation (CID) at Q2 to produce product or fragment ions. The previously-described mass spectrometry technique can also be referred to as multiple reaction monitoring, or MRM. In multiple reaction monitoring, both quadrupoles Q1 and Q3 can be fixed (or tuned) each at a single mass, whereas Q2 can serve as a collision cell.

The amount of each analyte (biomarker and internal standard) can be determined by comparing the area of precursor or product transitions, or both, with those of a standard calibration curve, e.g., a standard calibration curve generated from a series of defined concentrations of pure standards. Variables due to the extraction and the LC-MS/MS instrumentation can be normalized by normalizing peak areas of the analyte of interest to the peak areas of the internal standard. Any tandem MS machine and LC-MS/MS machine can be used, including the Quattro Premier LC-MS/MS system controlled by the PC-based MassLynx™ software.

A further aspect relates to a test kit for quantitating a biomarker directly in a biological sample, the kit comprising at least (i) a stock solution of an acid protease having a pH optimum at pH 1.0-5.0 (ii) an SPE column comprising sorbent designed for capture of at least one biomarker, being an immunoaffinity sorbent comprising an antibody against the at least one biomarker, and (iii) a labelled analog of said biomarker as internal standard. Preferably, the kit comprises pepsin. The labelled analog (for use as internal standard) is preferably a deuterated version of the biomarker. More preferably, the SPE sorbent is an immunoaffinity sorbent conjugated to distinct antibodies, each antibody capable of capturing a distinct biomarker of interest. In a specific aspect, a combination of antibodies against testosterone, 17-hydroxy-progesterone, cortisol and 11-deoxycortisol is used. This is beneficial in the diagnosis of hirsutism and allows to identify the cause of hirsutism.
Optionally, the kit may further comprise one or more selected from the group consisting of an analytical column, buffers/solvents necessary for performing the online solid phase extraction and subsequent chromatographic separation. Typical buffers for SPE include 0.2% formic acid in methanol; methanol+isopropanol+water (8:1:1, by volume); 0.2% formic acid in water; 10 v % methanol in water+0.2% formic acid; and 10% methanol in water+0.2% ammonium hydroxide.

The kit optionally further comprises a neutralization buffer and/or a set of standards of said biomarker. Buffer ingredients may be present in a dry form which upon reconstitution with water yields a ready-to-use buffer. In one embodiment, the kit further comprises an analytical column designed for the specific analyte(s) of interest. Preferably, the kit furthermore comprises one or more solvents for use in SPE and/or chromatography. The kit may further comprise a multi-well sample plate, e.g. a 96-well plate with 0.5 mL, 1.1 mL (1.0 mL capped), or 2.2 mL (2.0 mL capped) well volumes. The kit may contain instructions for use in an automated high-throughput method using (LC-(MS/)MS).

In one specific embodiment, the invention provides a test kit for quantitating at least one vitamin D derivative directly in a biological sample, the kit comprising at least (i) an acid protease stock solution (ii) an SPE column comprising an antibody against the vitamin D derivative, and (iii) a labelled analog of said at least one vitamin D derivative. Preferably, the kit comprises (i) pepsin, (ii) an SPE column comprising spherical particles with an immobilized antibody against the vitamin D derivative, e.g. 25011 Vitamin D3, (iii) stock solutions of 25-hydroxyvitamin D3, 25-hydroxyvitamin D2, 1α,25-dihydroxyvitamin D2 and/or 1α,25-dihydroxyvitamin D3, as unlabelled and as deuterated version. Deuterated vitamins D are preferably of the d6 form (deuterated in positions 26 and 27).

In one specific embodiment, the invention provides a test kit for quantitating testosterone directly in a biological sample, the kit comprising at least (i) an acid protease stock solution (ii) an SPE column comprising an immunoaffinity sorbent comprising an antibody against testosterone, (iii) a labelled testosterone. For example, a kit for detecting testosterone comprises (i) pepsin; (ii) an SPE column comprising spherical particles with immobilized antibody directed against testosterone; (iii) stock solution of testosterone, as unlabelled and as deuterated version; (iv) buffers necessary for performing the online solid phase extraction and (v) an analytical column, preferably a C18 column, such as HALO C18 2.1×100 mm, 2.7 μm commercially available from Advanced Materials Technology.

In another specific embodiment, the invention provides a kit for detecting melatonine, comprising (i) pepsin, (ii) an SPE column comprising spherical particles immobilized with antibody directed against melatonin; stock solution of melatonin, as unlabelled and as deuterated version; (iv) buffers necessary for performing the online solid phase extraction and (v) an analytical column, e.g. HALO C18 2.1×100 mm, 2.7 μm from Advanced Materials Technology.

In a further specific embodiment, the invention provides a test kit for the simultaneous quantitation of a vitamin D derivative and one or more metanephrines in a single biological sample, the kit comprising at least (i) an acid protease stock solution (ii) an SPE column comprising an immunoaffinity sorbent comprising one or more antibodie(s) against the vitamin D analog and metanephrines, (iii) labelled vitamin D analog and labelled metanephrine(s). For example, a kit for detecting metanephrines comprises (i) pepsin; (ii) an SPE column comprising spherical particles with immobilized antibody directed against 25OH Vitamin D and an immobilized antibody against one or more selected from metanephrine, normetanephrine and 3-methoxytyramine; stock solution of 25 OH vitamin D and metanephrine(s), as unlabelled and as deuterated version; (iv) buffers necessary for performing the online solid phase extraction and (v) an analytical column, preferably comprising functionalized ethylene bridged hybrid (BEH) particles, such as XSelect CSH Fluoro-Phenyl 2.0×100 mm, 2.5 μm commercially available from Waters.

In a further specific embodiment, the invention provides a test kit for quantitating one or more steroids (eg. testosterone, 17-hydroxyprogesterone, dehydroepiandrosterone sulphate, cortisol and 11-deoxycortisol) in a biological sample, the kit comprising at least (i) an acid protease stock solution (ii) an SPE column comprising an immunoaffinity sorbent comprising one or more antibodie(s) against the mentioned steroids, (iii) labelled steroids. For example, a kit for detecting these steroids comprises (i) pepsin; (ii) an SPE column comprising spherical particles with immobilized antibody directed against one or more selected from testosterone, 17-hydroxyprogesterone, cortisol and 11-deoxycortisol; (iii) stock solution of these steroid(s), as unlabelled and as deuterated version; (iv) buffers necessary for performing the online solid phase extraction and (v) an analytical column, preferably comprising functionalized ethylene bridged hybrid (BEH) particles, such as XSelect C18 2.1×100 mm, 2.5 μm commercially available from Waters.
The methods and kits described herein can be used in various diagnostic applications to monitor biomarker-related pathologies, biomarker homeostasis, and biomarker replacement therapies. For example, the total amount of dihydroxyvitamin D2 and/or D3 in a sample, such as a human patient sample, can be compared with clinical reference values to diagnose a vitamin D deficiency or hypervitaminosis D. Vitamin D deficiency in children will cause growth retardation and classic signs and symptoms of rickets. In adults, Vitamin D deficiency will precipitate and exacerbate both osteopenia and osteoporosis and increase the risk of fracture. Vitamin D deficiency has also been associated with increased risk of common cancers, autoimmune diseases, hypertension, infectious diseases, higher risk of upper respiratory tract infection and cardiovascular disease (Holick, Nature Reviews Endocrinology 7: 73-75 (2011); Holick et al., Am J Clin Nutr 87(suppl): 1080S-6S (2008)).
In one embodiment, a method and kit for determining whether or not a mammal has a vitamin D deficiency is provided. The method can involve determining the amount of 1α,25-dihydroxyvitamin D2 and/or 1α,25-dihydroxyvitamin D3 in a sample from the mammal, such as a human. The amounts can be determined using any of the methods provided herein. In another embodiment, a method for determining whether or not a mammal has hypervitaminosis D is provided. The method can involve determining the amount of 1α,25-dihydroxyvitamin D2 and/or 1α,25-dihydroxyvitamin D3 in a sample from the mammal using any of the methods described herein.

In another embodiment, a method and kit for determining testosterone in a mammal is provided. For example, it can be used for determining whether or not a mammal has a testosterone deficiency and/or to monitor the efficacy of testosterone replacement therapy. Testosterone deficiency (TD) afflicts approximately 30% of men aged 40-79 years, with an increase in prevalence strongly associated with aging and common medical conditions including obesity, diabetes, and hypertension (Traish et al., Am J Medicine 124: 578-587 (2011)). Decreased testosterone levels have been associated with an increase in all cause mortality as well as an increase in the incidence of classic cardiovascular risk factors including atherosclerosis, visceral obesity, insulin resistance, dyslipidemia, and hypertension, most of which are key components of the metabolic syndrome (Traish et al., Am J Medicine 124: 578-587 (2011); Ullah et al., Horm Metab Res 43: 153-164 (2011)).

Multi-analyte detection finds many clinical applications. For example, detection of multiple steroids may be used for the improved diagnosis of hirsutism. The major sign of hirsutism is coarse and pigmented body hair, appearing on the body where women don't commonly have hair—primarily the face, chest and back. Hirsutism is a common endocrinological complaint. The causes of this complaint can vary from dissatisfaction with a normal pattern of hair growth on the one hand, to the first clinical manifestation of androgen overproduction by an adrenal adenocarcinoma on the other. As there are several causes for hirsutism, the task inherent in the differential diagnosis of hirsutism is to determine whether the hirsutism is androgen medicated, whether the site of excess production of androgen is the adrenal gland or the ovary, and in most cases, whether the overproduction of androgen is dependent upon trophic hormone support, i.e. ACTH or LH and FSH. When this is done, appropriate therapy can be prescribed. By incorporating several steroids in one profile diagnosis can be simplified. (D. L. Loriaux, J Clin Endocrinol Metab 97(9): 2957-2968 (2012)).

### LEGEND TO THE FIGURES

FIG. 1: Calibration curve obtained by injecting dialyzed plasma treated with pepsin spiked with 25-OHVD3. Values on the Y-axis represent the ratio between signal of native 25-OHVD3 and signal of deuterated standard.

FIG. 2: Multiple reaction monitoring (MRM) chromatograms of 25-OHVD3 and its internal standard.

a) represents a plasma sample with a 25-OHVD3 concentration of 8.3 nmol/L. The upper panel shows the MRM of native 25-OHVD3, the lower panel shows the MRM of the hexa-deuterated internal standard of 25-OHVD3.

b) shows a plasma sample with 25-OHVD3 concentration of 165 nmol/L. Chromatogram (upper panel) shows MRM of native 25-OHVD3, lower panel shows the hexa-deuterated internal standard of 25-OHVD3

FIG. 3: Calibration curve obtained by injecting plasma samples spiked with testosterone.

FIG. 4: Scatter plot of the Passing and Bablok fit for the comparison of the liquid-liquid extraction versus the pepsin sample treatment.

FIG. 5: Chromatograms of total testosterone in a male and female plasma sample.

a) represents a male plasma sample with a testosterone concentration of 15.9 nmol/L (S/N=signal to noise ratio). Chromatograms shows the MRM of native testosterone (upper panel) and the tri-deuterated testosterone (lower panel)

b) shows a female plasma sample with a testosterone concentration of 0.082 nmol/L (S/N=signal to noise ratio). Chromatograms shows the MRM of native testosterone (upper panel) and the tri-deuterated testosterone (lower panel).

FIG. 6: Overlapped multiple reaction monitoring chromatograms of the ion suppression experiment of a pepsin treated plasma sample (solid line) and an untreated plasma sample (dotted line).

FIG. 7: Comparison of the immunoaffinity SPE method versus C₈-SPE method. Panel A: Chromatograms of total testosterone in a female plasma sample analyzed according to the protocol in example 3; Panel B: Chromatograms of total testosterone in a female plasma sample analyzed according to the protocol in comparative example 2.

### EXPERIMENTAL SECTION

### Example 1

### Automated High-Throughput Method for the Determination of 25-Hydroxyvitamin D3

### Materials

25-hydroxyvitamin D₃ (25 OHVD3) was purchased from Sigma Aldrich (St. Louis, Mo.). A certified 25-hydroxyvitamin D₃ reference standard from the Nation Institute of Standards and Technology was used to correct for impurity of the Sigma material (correction factor).
25OHD3 was reconstituted in methanol and stored at −20° C. (stock solution). Working standards were prepared by diluting the stock solution in dialyzed EDTA-plasma (treated with pepsin) with concentrations of 0-200 nmol/L. Deuterated d₆-25 OHVD3 was purchased from Medical Isotopes (USA) for use as an internal standard. The compound was reconstituted in methanol and stored at −20° C. A working internal standard solution was created by serial dilution to ˜0.4 μmol/L in methanol. Pepsin (≧2,500 units/mg protein) was purchased from Sigma Aldrich (St. Louis, Mo.). A working solution of 425 mg pepsin in 100 mL 1M hydrochloric acid was prepared and stored at 4° C. until use.

### Sample Preparation:

Pipette 500 μL sample into a 96-deep-well plate. Add 50 μL pepsin solution to each sample, cover the well plate and shake for 1 min. Incubate for 1 hour at room temperature to allow the pepsin to digest the plasma proteins. After the incubation time, add 100 μL of internal standard to all samples and wells for the standards. Pipette to each respective well the respective volume of standard working solution. Fill up the volume to 1.0 mL with vitamin C/phosphoric acid solution. Place the 96-deep-well-plate into the autosampler.

### Online Extraction and LC-MS/MS

Online extraction and HPLC chromatography of the standards and samples was performed using a Spark Holland Symbiosis® online SPE system. This system consists of a temperature-controlled autosampler (temperature maintained at 6° C.), a SPE controller unit (automated cartridge exchanger), a solvent delivery unit (2 high pressure dispensers), and an HPLC pump. For a more detailed description of this system see De Jong et al. (Clin Chem 2007 53: 1684-1693).

The SPE cartridges were packed with an immunoaffinity sorbent containing immobilized antibodies raised against Vitamin D (see e.g. Kobayashi et al. J. Steroid Biochem. Molec. Biol. 1994; 48:567-572). The analytical column was a HALO C18 2.1×100 mm, 2.7 μm from Advanced Materials Technology. 100 μL of the standards or samples was injected onto the SPE cartridge. Scheme 1 shows the equilibration, wash and elution steps of the online SPE procedure+the LC procedure and the MS/MS parameters.

Scheme 1:

| | HPD | port | solvent |
|---|---|---|---|
| HPD (High | HPD 1 (right) | SSM port 1A | Acetonitrile (ACN) |
| Pressure | | SSM port 1B | Ultrapure Water |
| Dispensers) | | SSM port 1C | 0.1% FA in Ultrapure water |
| | | SSM port 1D | 25 mM ammonium acetate, pH 7.2 |
| | | SSM port 1E | 0.1% FA in 20% ACN |
| | | SSM port 1F | Not used |
| | | valve port 3 | Not used |
| | HPD2 (left) | valve port 1 | ACN |
| | | valve port 2 | Ultrapure Water |
| | | valve port 3 | 0.1% FA in 20% ACN |
| | | valve port 4 | 25 mM ammonium acetate, pH 7.2 |

| SPE | cartridge | Immunoaffinity cartridges - vitamin D | | | |
|---|---|---|---|---|---|
| | type | | | | |
| | Automated Soild Phase Extraction Procedure | | | | |
| | | | | | MIX |
| | Action | volume | flow | solvent | ratio |
| | Conditioning | 1000 μl | 2500 μl/min | 0.1% FA in | |
| | (1) | | | Ultrapure water | |
| | Equilibration | 1000 μl | 2500 μl/min | 25 mM ammonium | |
| | (1) | | | acetate, pH 7.2 | |
| | Equilibration | 1000 μl | 2500 μl/min | 25 mM ammonium | |
| | (2) | | | acetate, pH 7.2 | |
| | sample | 1000 μl | 500 μl/min | 25 mM ammonium | |
| | extraction | | | acetate, pH 7.2 | |
| | wash step (1) | 2000 μl | 2500 μl/min | 25 mM ammonium | |
| | | | | acetate, pH 7.2 | |
| | wash step (2) | 2000 μl | 2500 μl/min | Ultrapure water | |
| | wash step (3) | 500 μl | 2500 μl/min | 0.1% FA in 10% | |
| | | | | Methanol | |
| | clamp flush (1) | 1000 μl | 2500 μl/min | 0.1% FA in 20% | |
| | | | | Methanol | |
| | clamp flush (2) | 1500 μl | 2500 μl/min | Ultrapure water | |
| | clamp flush (3) | 1000 μl | 5000 μl/min | 25 mM ammonium | |
| | | | | acetate, pH 7.2 | |
| | elution | 300 μl | 300 μl/min | mobile phase (through LC | |
| | | | | pump) | |
| | elution mode | standard | Elution | 1.00 min | |
| | | | time | | |

| | Note: all solvents were pre-mixed | | | | | |
|---|---|---|---|---|---|---|
| | LC parameters | | | | | |
| LC pump | solvent A | 10 mM ammonium acetate + 0.1% FA in methanol/water | | | | |
| (dual pump) | | (80/20) | | | | |
| | solvent B | 0.1% FA in Methanol | | | | |
| | solvent mode | gradient | | | | |
| | flow | 0.3 ml/min | | | | |
| | Total runtime | 9.5 min | | | | |
| | Degasser | On | | | | |
| | gradient- | time | flow | solvent | solvent | gradient |
| | program | (mm:ss) | (ml/min) | A % | B % | type |
| | | 00:01 | 0.3 | 80 | 20 | |
| | | 05:00 | 0.3 | 0 | 100 | Linear |
| | | 06:00 | 0.3 | 0 | 100 | Isocratic |
| | | 06:30 | 0.3 | 80 | 20 | Linear |
| | | 09:30 | 0.3 | 80 | 20 | Isocratic |

MS-MS system parameters

| Detection | System | Quattro Premier (XE) | | |
|---|---|---|---|---|
| (MS-MS) | tune page | source | ion mode | tune page settings |
| | settings | | capillary voltage | 1 kV |
| | | | cone voltage | Variable |
| | | | extractor voltage | 3 V |
| | | | RF lens voltage | 0.1 V |
| | | | source | 150° C. |
| | | | temperature | |
| | | | desolvation | 500° C. |
| | | | temperature | |
| | | | desolvation gas | 1100 L/hr |
| | | | flow | |
| | | | cone gas flow | 150 L/hr |
| | | analyser 1 | LM resolution 1 | 13.3 |
| | | | HM resolution 1 | 13.3 |
| | | | ion energy 1 | 0.1 |
| | | collision | Argon flow | 0.25 mL/min |
| | | cell | entrance | 0 |
| | | | collision energy | variable (eV) |
| | | | exit | 0 |
| | | analyser 2 | LM resolution 2 | 13.3 |
| | | | HM resolution 2 | 13.2 |
| | | | ion energy 2 | 0.2 |
| | | multiplier | 650 | |

| parent | daughter | dwell | Cone | collision |
|---|---|---|---|---|
| m/z | m/z | time | Voltage | energy |
| SRM Quantifiers | | | | |
| 401.4 | 257.1 | 0.05 sec | 16 V | 15 eV |
| 407.4 | 263.1 | 0.05 sec | 16 V | 15 eV |
| SRM Qualifiers | | | | |
| 383.4 | 257.1 | 0.05 sec | 25 V | 14 eV |
| 389.4 | 263.1 | 0.05 sec | 25 V | 16 eV |

Analytes were analyzed on a Waters Quattro® Premier tandem mass spectrometer equipped with a Z Spray® ion source operated in positive electrospray ionization mode. All aspects of system operation and data acquisition were controlled using MassLynx v4.1 software with automated data processing using the TargetLynx Application Manager (Waters).

### Results:

TABLE 1

| Recovery ± | | | | | | | |
|---|---|---|---|---|---|---|---|
| SD | Precision: mean ± CV (%) | | | Linear | | | |
| (%)* | QC | intra-assay | inter-assay | range | LOD | LOQ | Unit |
| 93.5 ± 0.7 | Low | 21.6 ± 7.2% | 25.1 ± 10.5% | 0-280 | 1.2 | 4.0 | nmol/l |
| 97.8 ± 0.7 | Medium | 53.3 ± 5.0% | 56.5 ± 11.6% | | | | |
| 97.9 ± 0.4 | High | 145.5 ± 6.3% | 150.3 ± 14.1% | | | | |

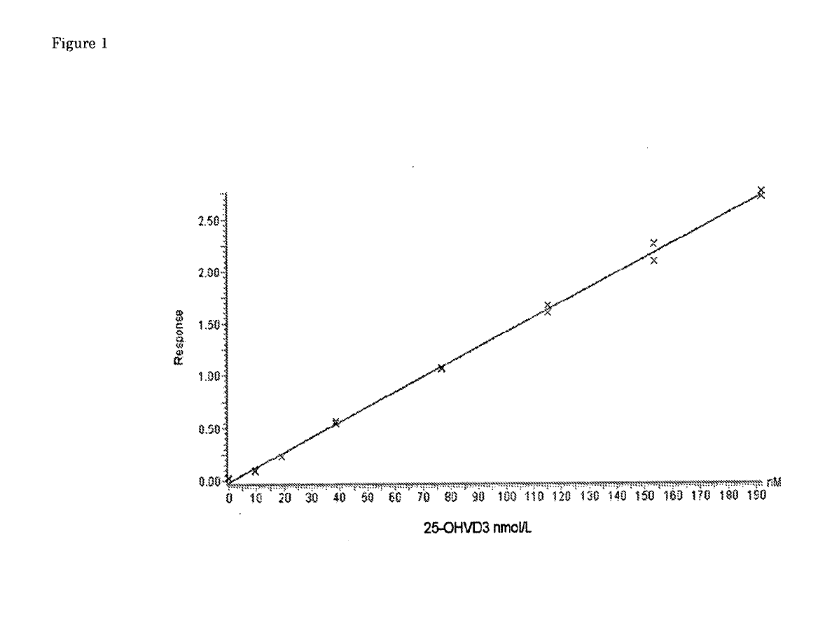

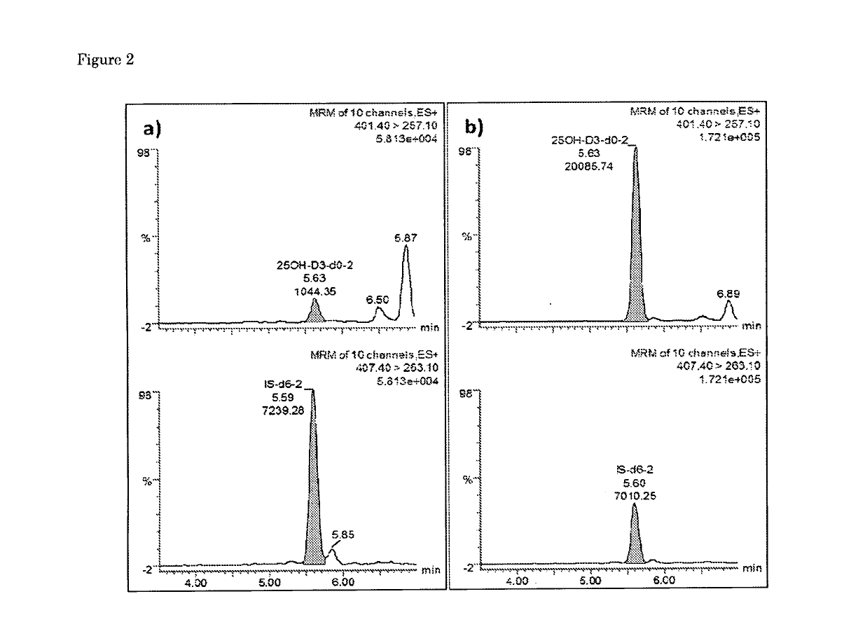

FIG. 1 shows a typical calibration curve of 25-OHVD3, correlation coefficient >0.99. Recovery was determined by looking at the in-line recovery. This in-line recovery was performed by switching two cartridges in-line (in sequence). The second cartridge will capture the breakthrough on the first cartridge. This method is programmable by the MassLynx software (Advanced Method Development). The three QC samples were analyzed to check on the breakthrough and recovery. Recovery was close to 100%, so breakthrough is minimal (almost no analyte loss).

Intra- and interassay variation was determined by use of three QC-samples with low, medium and high levels of 25-OHVD3. Intra-assay imprecision was obtained by measuring 20 replicates in a single series. The inter-assay imprecision was obtained from 20 different assays over a 3-week period. Intra-assay CV (n=20) was 5.0 to 7.2%. Inter-assay CV (n=20) was 10.5 to 14.1%.

Linearity was determined by plotting the ratios of analyte peak area to internal standard peak area against 25-OHVD3 at 8 concentrations in the intervals 0 to 280 nmol/L. On 20 different days, fresh calibration lines were prepared and measured. Plasma calibration curves were reproducible between days, with R²>0.99. Limit of detection (LOD) and limit of quantitation (LOQ) were determined by the MassLynx Software. LOD was defined as signal to noise ratio=3 and LOQ was defined as signal to noise ratio=4.0 nmol/L,

### Example 2

### Testosterone Detection (Comparative Example)

### Materials

Testosterone was purchased from Sigma Aldrich (St. Louis, Mo.), reconstituted in methanol and stored at −20° C. (stock solution). Working standards were prepared by diluting the stock solution in dialyzed EDTA-plasma (treated with pepsin) with concentrations of 0-60 nmol/L. Deuterated d₃-testosterone was purchased from C/D/N/ Isotopes Inc. (Canada) for use as an internal standard. The compound was reconstituted in methanol and stored at −20° C. A internal standard working solution was created by serial dilution to ˜20 nmol/L in methanol. Pepsin (≧2,500 units/mg protein) was purchased from Sigma Aldrich (St. Louis, Mo.). A working solution of 425 mg pepsin in 100 mL 1M hydrochloric acid was prepared and stored at 4° C. until use.

### Sample Preparation:

Pipette 250 μL sample into the respective vials. Add 50 μL internal standard working solution to each sample. Add 25 μL pepsin solution to each sample, close the vials and vortex. Incubate for 1 hour at room temperature to allow the pepsin to digest the plasma proteins. In the meantime pipette to each respective vial the respective volume of standard working solution and internal standard working solution. After the incubation time fill up the volume to 1.0 mL with ultrapure water. Place the vials into the autosampler.

### Online Extraction and LC-MS/MS

Online extraction and HPLC chromatography of the standards and samples was performed using a Spark Holland Symbiosis® online SPE system. This system consists of a temperature-controlled autosampler (temperature maintained at 6° C.), a SPE controller unit (automated cartridge exchanger), a solvent delivery unit (2 high pressure dispensers), and an HPLC pump. For a more detailed description of this system see De Jong et al. (Plasma free metanephrine measurement using automated online-solid-phase extraction HPLC-tandem mass spectrometry Clin Chem 2007 53: 1684-1693).

The cartridges that were used for the SPE were HySphere C₈ EC-SE, 10 μm from Spark Holland. The analytical column was a HALO C18 2.1×100 mm, 2.7 μm from Advanced Materials Technology. 100 μL of the standards or samples was injected onto the HySphere C₈ cartridge. Scheme 2 shows the equilibration, wash and elution steps of the online SPE procedure+the LC procedure and the MS/MS parameters.

Analytes were analyzed on a Waters Quattro® Premier tandem mass spectrometer equipped with a Z Spray® ion source operated in positive electrospray ionization mode. All aspects of system operation and data acquisition were controlled using MassLynx v4.1 software with automated data processing using the TargetLynx Application Manager (Waters).

Scheme 2:

| | HPD | port | solvent |
|---|---|---|---|
| HPD (High | HPD 1 | SSM port 1A | Acetonitrile (ACN) |
| Pressure | (right) | SSM port 1B | Ultrapure Water |
| Dispensers) | | SSM port 1C | 4 mM ammonium acetate in ACN/water |
| | | | (2/8) + 0.1% FA |
| | | SSM port 1D | 4 mM ammonium acetate in ACN/water |
| | | | (2/8) + 0.2% ammonium hydroxide |
| | | SSM port 1E | 4 mM ammonium acetate in ACN/water |
| | | | (1/9) + 0.1% FA |
| | | SSM port 1F | Not used |
| | | valve port 3 | Not used |
| | HPD2 (left) | valve port 1 | ACN |
| | | valve port 2 | Ultrapure Water |
| | | valve port 3 | 4 mM ammonium acetate in ACN/water |
| | | | (2/8) + 0.1% FA |
| | | valve port 4 | Not used |

| SPE | cartridge | HySphere C8 EC-SE, 10 μm (Spark Holland) | | | |
|---|---|---|---|---|---|
| | type | | | | |
| | Automated Solid Phase Extraction Procedure | | | | |
| | | | | | MIX |
| | Action | volume | flow | solvent | ratio |
| | Conditioning (1) | 1000 μl | 5000 μl/min | ACN | |
| | Equilibration (1) | 1000 μl | 5000 μl/min | Ultrapure Water | |
| | Equilibration (2) | 1000 μl | 5000 μl/min | 4 mM ammonium | |
| | | | | acetate in | |
| | | | | ACN/water (1/9) + | |
| | | | | 0.1% FA | |
| | sample extraction | 1000 μl | 2000 μl/min | 4 mM ammonium | |
| | | | | acetate in | |
| | | | | ACN/water (1/9) + | |
| | | | | 0.1% FA | |
| | wash step (1) | 1250 μl | 5000 μl/min | 4 mM ammonium | |
| | | | | acetate in | |
| | | | | ACN/water (2/8) + | |
| | | | | 0..2% ammonium | |
| | | | | hydroxide | |
| | wash step (2) | 2000 μl | 5000 μl/min | 4 mM ammonium | |
| | | | | acetate in | |
| | | | | ACN/water (1/9) + | |
| | | | | 0.1% FA | |
| | clamp flush (1) | 1000 μl | 5000 μl/min | 4 mM ammonium | |
| | | | | acetate in | |
| | | | | ACN/water (2/8) + | |
| | | | | 0.1% FA | |
| | clamp flush (2) | 1500 μl | 5000 μl/min | ACN | |
| | clamp flush (3) | 1000 μl | 5000 μl/min | Ultrapure Water | |
| | elution | 300 μl | 300 μl/min | mobile phase (through LC | |
| | | | | pump) | |
| | elution mode | standard | Elution | 1.30 min | |
| | | | time | | |

Note: all solvents were pre-mixed

LC parameters

| LC pump | solvent A | 2 mM ammonium acetate + 0.1% FA in water | | | | |
|---|---|---|---|---|---|---|
| (dual pump) | solvent B | 0.1% FA in ACN/water (95/5) | | | | |
| | solvent mode | gradient | | | | |
| | flow | 0.2 ml/min | | | | |
| | Total runtime | 8.5 min | | | | |
| | Degasser | On | | | | |
| | gradient- | time | flow | solvent | solvent | gradient |
| | program | (mm:ss) | (ml/min) | A % | B % | type |
| | | 00:01 | 0.2 | 70 | 30 | |
| | | 04:00 | 0.2 | 30 | 70 | Linear |
| | | 05:00 | 0.2 | 30 | 70 | Isocratic |
| | | 05:30 | 0.2 | 70 | 30 | Linear |
| | | 08:30 | 0.2 | 70 | 30 | Isocratic |

MS-MS system parameters

| Detection (MS- | System | Quattro Premier (XE) | | |
|---|---|---|---|---|
| MS) | tune page | source | ion mode | ESI+ |
| | settings | | capillary voltage | 1 kV |
| | | | cone voltage | 30 |
| | | | extractor voltage | 4 V |
| | | | RF lens voltage | 0.5 V |
| | | | source | 150° C. |
| | | | temperature | |
| | | | desolvation | 500° C. |
| | | | temperature | |
| | | | desolvation gas | 1100 L/hr |
| | | | flow | |
| | | | cone gas flow | 150 L/hr |
| | | analyser 1 | LM resolution 1 | 13.3 |
| | | | HM resolution 1 | 1.3.3 |
| | | | ion energy 1 | 0.1 |
| | | collision | Argon flow | 0.25 mL/min |
| | | cell | entrance | 0 |
| | | | collision energy | variable (eV) |
| | | | exit | 0 |
| | | analyser 2 | LM resolution 2 | 13.3 |
| | | | HM resolution 2 | 13.2 |
| | | | ion energy 2 | 0.2 |
| | | multiplier | 650 | |

| parent | daughter | dwell | cone | collision |
|---|---|---|---|---|
| m/z | m/z | time | voltage | energy |
| SRM Quantifiers | | | | |
| 289.1 | 97.1 | 0.10 sec | 30 V | 22 eV |
| 292.1 | 97.1 | 0.10 sec | 30 V | 22 eV |
| SRM Qualifiers | | | | |
| 289.1 | 109.1 | 0.10 sec | 30 V | 26 eV |
| 292.1 | 109.1 | 0.10 sec | 30 V | 26 eV |

### Results

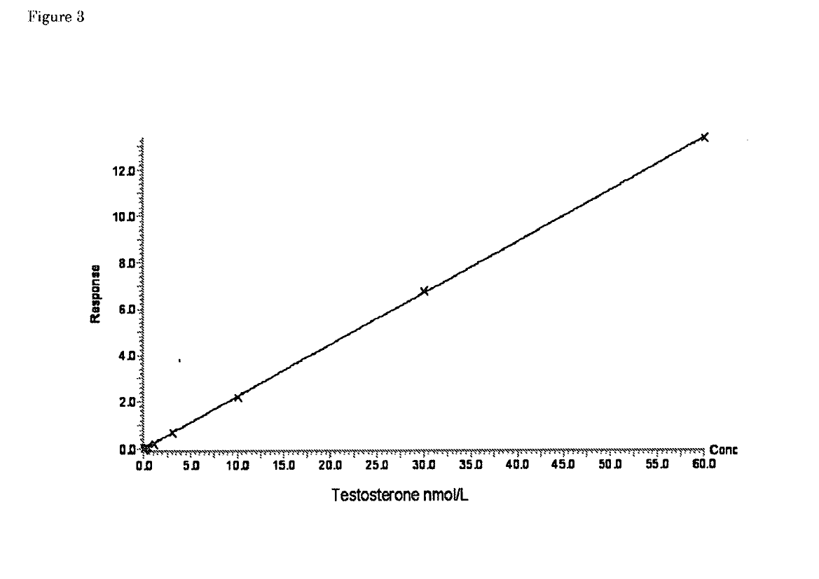

The calibration line was linear over the examined range with correlation coefficients >0.999. A representative example can be seen in FIG. 3. To see if the pepsin was able to release all of the bound testosterone, a comparison of the pepsin method was made with an already published method: Turpeinen et al. Scand J Clin Lab Investigation, 2008 68: 50-57. Here they used a liquid-liquid extraction to release all of the testosterone.
10 EDTA-Plasma Samples were Split and Measured in Duplicate by:
1) performing a liquid-liquid extraction according to the prior art and then analyzed by the method mentioned above
2) by performing the pepsin treatment according to the invention and analyzed by the method mentioned above
   Table 2 shows the concentrations of the analyzed samples for the LLE and pepsin method.

TABLE 2

| | | |
|---|---|---|
| Sample | LLE | Pepsin |
| # | nmol/L | nmol/L |

| | | | | |
|---|---|---|---|---|
| 1 | 0.575 | 0.596 | 0.536 | 0.621 |
| 2 | 0.433 | 0.411 | 0.35 | 0.365 |
| 3 | 1.574 | 1.646 | 1.637 | 1.643 |
| 4 | 0.38 | 0.421 | 0.386 | 0.418 |
| 5 | 2.707 | 2.706 | 2.865 | 2.923 |
| 6 | 4.741 | 4.854 | 4.844 | 4.75 |
| 7 | 4.051 | 3.986 | 3.665 | 3.731 |
| 8 | 3.629 | 3.656 | 3.743 | 3.745 |
| 9 | 8.399 | 8.245 | 8.428 | 8.212 |
| 10 | 10.936 | 10.443 | 10.684 | 10.755 |

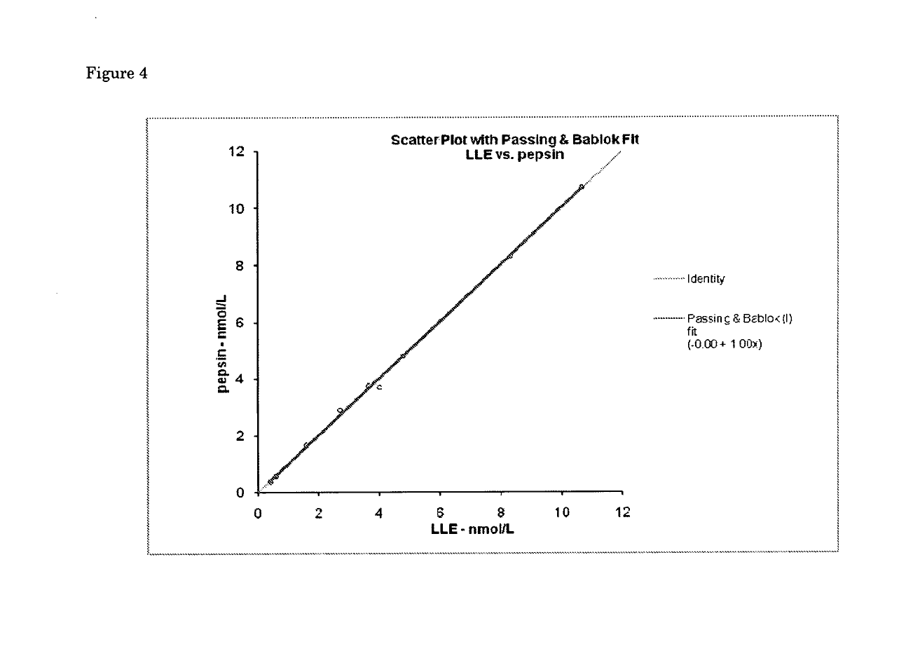

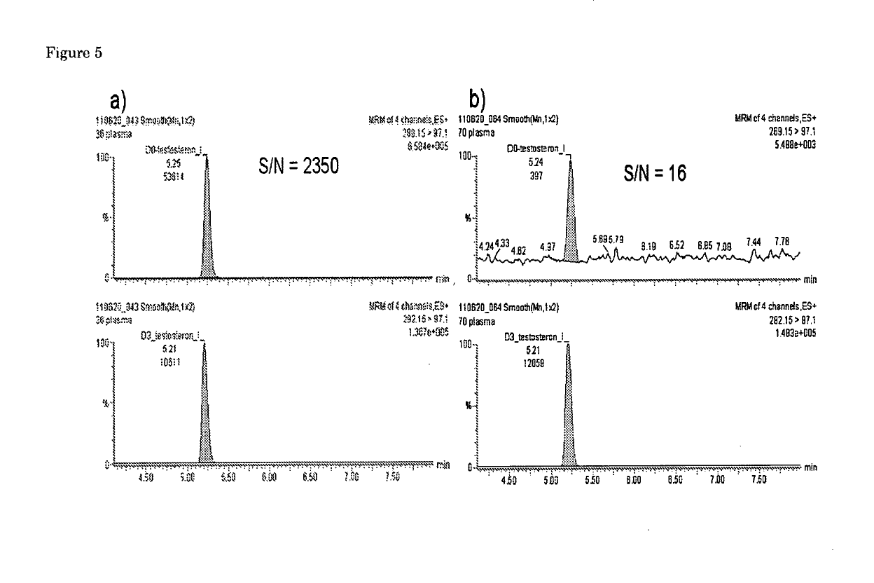

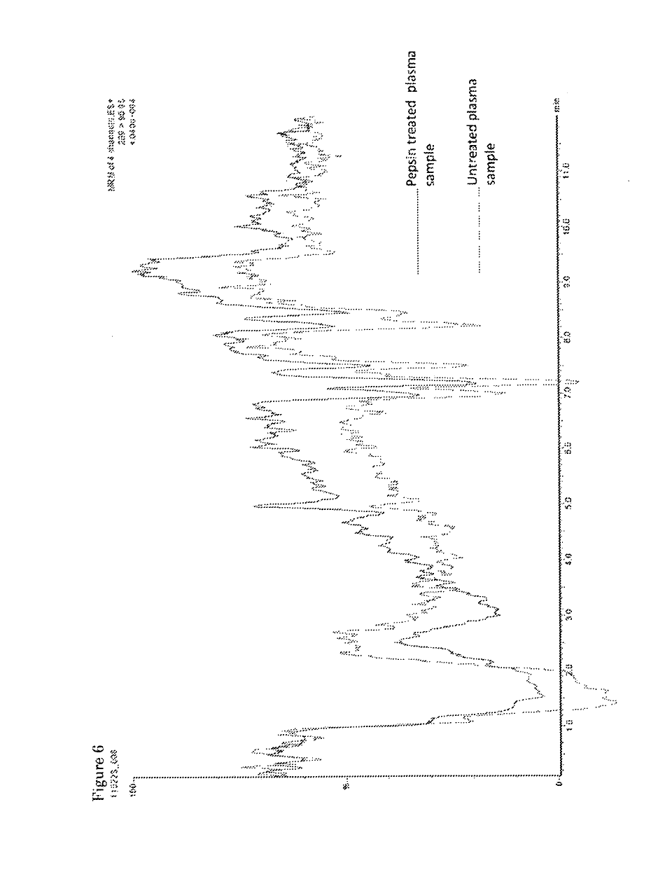

Passing-Bablok regression (see FIG. 4) was used to compare the two sample preparation methods. The intercept A is a measure of the systematic (constant) differences between the two methods. The 95% confidence interval (95% CI) for the intercept A can be used to test the hypothesis that A=0. This hypothesis is accepted if the confidence interval for A contains the value 0. If the hypothesis is rejected, then it is concluded that A is significant different from 0 and both methods differ at least by a constant amount. Our results show that the systematic difference is −0.05 to 0.09 (95% CI), so no significant difference.
The slope B is a measure of the proportional differences between the two methods. The 95% confidence interval for the slope B can be used to test the hypothesis that B=1. This hypothesis is accepted if the confidence interval for B contains the value 1. If the hypothesis is rejected, then it is concluded that B is significant different from 1 and there is at least a proportional difference between the two methods. Our results show that the proportional difference is 0.98 to 1.04, so it can be concluded that no proportional difference is present. The results clearly demonstrate that the acid protease treatment is capable of releasing the testosterone of its binding proteins just as efficient as the traditional liquid-liquid extraction procedure. Thus, the method of the invention provides a valuable alternative for the time-consuming and cumbersome method of the prior art.

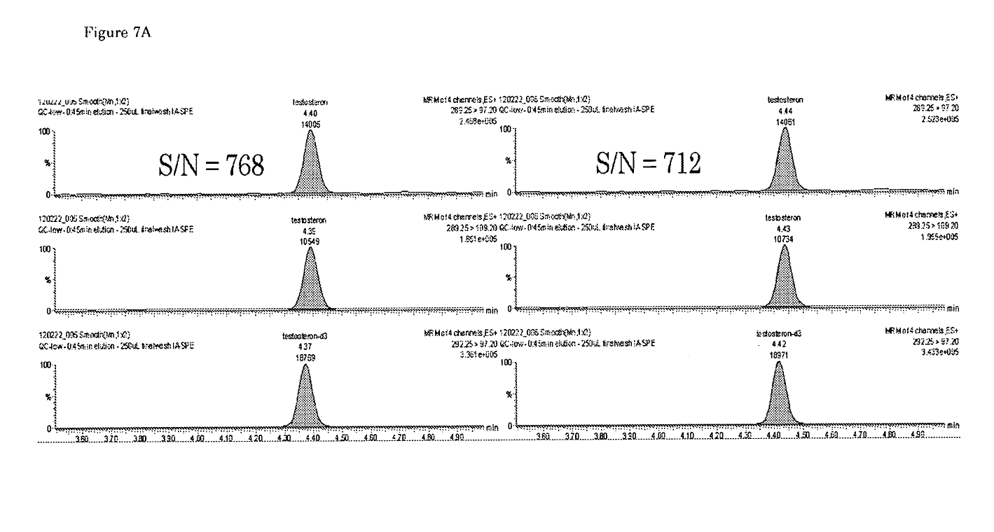

Subsequently, an ion-suppression study was performed to check the effect of the pepsin treatment on the ion-suppression (in comparison to an untreated sample). This was performed by infusing testosterone post-column (25 nmol/L) and performing a normal analysis on a analyzing a plasma sample according to the protocol mentioned in example 2 (treated with pepsin or untreated). See Annesly, Clin. Chem 49: 1041-1044 (2003) for a more detailed description. FIG. 6 shows the results of this experiment. The multiple reaction chromatograms were compared with each other by overlapping them. The first minute the signal of the two compared methods is the same, during this time the SPE extraction is performed, so only mobile phase enters the mass spectrometer. Then a sharp decrease in signal follows in both chromatograms, this is the solvent front. After this the difference in the two chromatograms is clearly visible. The untreated plasma sample shows a lower signal at the retention time where testosterone is analyzed.

Thus, pepsin treatment has no negative effect on the ion-suppression, but shows even less ion-suppression in comparison to the untreated plasma sample. This indicates a further, unexpected advantage of the method of the present invention.

### Example 3

### Testosterone Detection Using Immunoaffinity SPE

### Materials & Sample Preparation:

### See Example 2.

### Online Extraction and LC-MS/MS

Online extraction and HPLC chromatography of the standards and samples was performed using a Spark Holland Symbiosis® online SPE system. This system consists of a temperature-controlled autosampler (temperature maintained at 6° C.), a SPE controller unit (automated cartridge exchanger), a solvent delivery unit (2 high pressure dispensers), and an HPLC pump. For a more detailed description of this system see WO2008/144301.

The SPE cartridges were packed with an immunoaffinity sorbent containing immobilized antibodies raised against testosterone (J. J. Pratt, T. Wiegman, R. E. Lappöhn and M. G. Wöldring Estimation of plasma testosterone without extraction and chromatography, Clinica Chimica Acta 1975 59: 337-346). The analytical column was a HALO C18 2.1×100 mm, 2.7 μm from Advanced Materials Technology. 100 μL of the standards or samples was injected onto the cartridge. The equilibration, wash and elution steps of the online SPE procedure Scheme 1 of Example 1, with the exception that the immunoaffinity SPE cartridges contained an immobilized antibody against testosterone. The LC procedure and the MS/MS parameters were performed according to scheme 2 of Example 2.

Analytes were analyzed on a Waters Quattro® Premier tandem mass spectrometer equipped with a Z Spray® ion source operated in positive electrospray ionization mode. All aspects of system operation and data acquisition were controlled using MassLynx v4.1 software with automated data processing using the TargetLynx Application Manager (Waters).

### Results

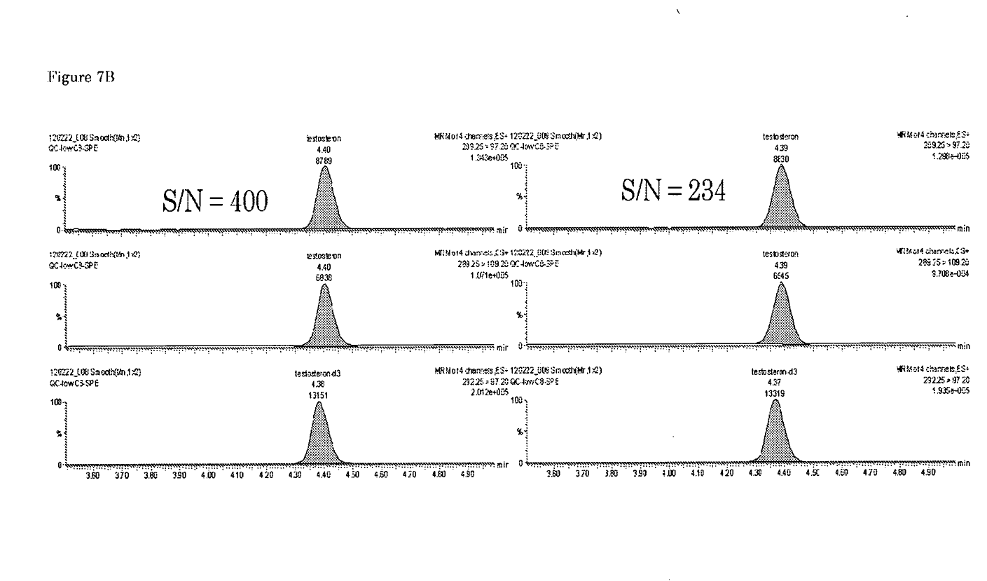

FIG. 7 a) shows the chromatograms of two separate analyses of a female plasma sample (total testosterone=1.6 nmol/L) according to the immunoaffinity protocol according to the invention (example 3). The same sample was also analyzed in duplicate with the protocol mentioned in example 2 (FIG. 7 b). The immunoaffinity protocol clearly shows improved signal to noise ratio (S/N) in comparison to the conventional C8 material. This indicates that with the immunoaffinity SPE step a better clean-up is achieved (less background). Also, the combination of the protein digestion step with the online immunoaffinity extraction is proven successful.

### Example 4

### Melatonin Detection

### Materials

Melatonin was purchased from Sigma Aldrich (St. Louis, Mo.), reconstituted in methanol and stored at −20° C. (stock solution). Working standards were prepared by diluting the stock solution in dialyzed EDTA-plasma (treated with pepsin) with concentrations of 10-5000 pmol/L. Deuterated d₄-melatonin was purchased from C/D/N/ Isotopes Inc. (Canada) for use as an internal standard. The compound was reconstituted in methanol and stored at −20° C. A internal standard working solution was created by serial dilution to ˜1 nmol/L in methanol. Pepsin (≧2,500 units/mg protein) was purchased from Sigma Aldrich (St. Louis, Mo.). A working solution of 425 mg pepsin in 100 mL 1M hydrochloric acid was prepared and stored at 4° C. until use.

### Sample Preparation:

Pipette 250 μL sample into the respective vials. Add 50 uL internal standard working solution to each sample. Add 25 μL pepsin solution to each sample, close the vials and vortex. Incubate for 1 hour at room temperature to allow the pepsin to digest the plasma proteins. In the meantime pipette to each respective vial the respective volume of standard working solution and internal standard working solution. After the incubation time neutralize by adding 25 μL, 1M sodium hydroxide solution and fill up the volume to 1.0 mL with ultrapure water. Place the vials into the autosampler.

### Online Extraction and LC-MS/MS

Online extraction and HPLC chromatography of the standards and samples was performed using a Spark Holland Symbiosis® online SPE system. This system consists of a temperature-controlled autosampler (temperature maintained at 6° C.), a SPE controller unit (automated cartridge exchanger), a solvent delivery unit (2 high pressure dispensers), and an HPLC pump. For a more detailed description of this system see WO2008/144301.

The cartridges that were used for the SPE were packed with an immunoaffinity sorbent containing immobilized antibodies raised against melatonin (M. D. Rollag and G. D. Nisweder, Radioimmunoassay of serum concentrations of melatonin in sheep exposed to different lighting regimens, Endocrinology 1976 98: 482-489).

The analytical column was a HALO C18 2.1×100 mm, 2.7 μm from Advanced Materials Technology. 100 μL of the standards or samples was injected onto the cartridge. Scheme 4 shows the equilibration, wash and elution steps of the online SPE procedure+the LC procedure and the MS/MS parameters.

Scheme 4:

| | HPD | port | solvent |
|---|---|---|---|
| HPD (High | HPD 1 | SSM port 1A | Acetonitrile (ACN) |
| Pressure | (right) | SSM port 1B | Ultrapure Water |
| Dispensers) | | SSM port 1C | 0.1% FA in Ultrapure water |
| | | SSM port 1D | 25 mM ammonium acetate, pH 7.2 |
| | | SSM port 1E | 0.1% FA in 10% ACN |
| | | SSM port 1F | Not used |
| | HPD2 (left) | valve port 3 | Not used |
| | | valve port 1 | ACN |
| | | valve port 2 | Ultrapure Water |
| | | valve port 3 | 0.1% formic acid in 20% ACN |
| | | valve port 4 | 25 mM ammonium acetate, pH 7.2 |

| | | |
|---|---|---|
| SPE | cartridge | Immunoaffinity cartridges - melatonin |
| | type | |

| Automated Solid Phase Extraction Procedure | | | | |
|---|---|---|---|---|
| | | | | MIX |
| Action | volume | flow | solvent | ratio |
| Conditioning | 1000 μl | 2500 μl/min | 0.1% FA in | |
| (1) | | | Ultrapure water | |
| Equilibration | 1000 μl | 2500 μl/min | 25 mM ammonium | |
| (1) | | | acetate, pH 7.2 | |
| Equilibration | 1000 μl | 2500 μl/min | 25 mM ammonium | |
| (2) | | | acetate, pH 7.2 | |
| sample | 1000 μl | 500 μl/min | 25 mM ammonium | |
| extraction | | | acetate, pH 7.2 | |
| wash step | 2000 μl | 2500 μl/min | 25 mM ammonium | |
| (1) | | | acetate, pH 7.2 | |
| wash step (2) | 2000 μl | 2500 μl/min | Ultrapure water | |
| wash step (3) | 500 μl | 2500 μl/min | 0.1% FA in 10% | |
| | | | ACN | |
| clamp flush | 1000 μl | 2500 μl/min | 0.1% FA in 20% | |
| (1) | | | ACN | |
| clamp flush | 1500 μl | 2500 μl/min | Ultrapure water | |
| (2) | | | | |
| clamp flush | 1000 μl | 5000 μl/min | 25 mM ammonium | |
| (3) | | | acetate, pH 7.2 | |
| elution | 300 μl | 300 μl/min | mobile phase (through LC | |
| | | | pump) | |
| elution | standard | Elution | 1.00 min | |
| mode | | time | | |

Note: all solvents were pre-mixed

LC parameters

| LC pump | solvent A | 2 mM ammoniumacetaat + 0.1% FA in water | | | | |
|---|---|---|---|---|---|---|
| (dual pump) | solvent B | 0.1% FA in ACN/water (95/5) | | | | |
| | solvent | gradient | | | | |
| | mode | | | | | |
| | flow | 0.2 ml/min | | | | |
| | Total | 8.5 min | | | | |
| | runtime | | | | | |
| | Degasser | On | | | | |
| | gradient- | time | flow | solvent | solvent | gradient |
| | program | (mm:ss) | (ml/min) | A % | B % | type |
| | | 00:01 | 0.2 | 70 | 30 | |
| | | 04:00 | 0.2 | 30 | 70 | Linear |
| | | 05:00 | 0.2 | 30 | 70 | Isocratic |
| | | 05:30 | 0.2 | 70 | 30 | Linear |
| | | 08:30 | 0.2 | 70 | 30 | Isocratic |

MS-MS system parameters

| Detection | System | Quattro Premier (XE) | | |
|---|---|---|---|---|
| (MS-MS) | tune page | source | ion mode | ESI+ |
| | settings | | capillary voltage | 1 kV |
| | | | cone voltage | 30 |
| | | | extractor voltage | 4 V |
| | | | RF lens voltage | 0.5 V |
| | | | source | 150° C. |
| | | | temperature | |
| | | | desolvation | 500° C. |
| | | | temperature | |
| | | | desolvation gas | 1100 L/hr |
| | | | flow | |
| | | | cone gas flow | 150 L/hr |
| | | analyser 1 | LM resolution 1 | 13.3 |
| | | | HM resolution 1 | 13.3 |
| | | | ion energy 1 | 0.1 |
| | | collision | Argon flow | 0.25 mL/min |
| | | cell | entrance | 0 |
| | | | collision energy | variable (eV) |
| | | | exit | 0 |
| | | analyser 2 | LM resolution 2 | 13.3 |
| | | | HM resolution 2 | 13.2 |
| | | | ion energy 2 | 0.2 |
| | | multiplier | 650 | |

| | daughter | dwell | cone | collision |
|---|---|---|---|---|
| parent m/z | m/z | time | voltage | energy |
| SRM Quantifiers | | | | |
| 233.0 | 174.0 | 0.107 sec | 21 V | 14 eV |
| 237.0 | 178.0 | 0.107 sec | 21 V | 14 eV |
| SRM Qualifiers | | | | |
| 233.0 | 159.0 | 0.107 sec | 21 V | 27 eV |
| 237.0 | 163.0 | 0.107 sec | 21 V | 27 eV |

Analytes were analyzed on a Waters Quattro® Premier tandem mass spectrometer equipped with a Z Spray® ion source operated in positive electrospray ionization mode. All aspects of system operation and data acquisition were controlled using MassLynx v4.1 software with automated data processing using the TargetLynx Application Manager (Waters).

### Example 5

### Automated High-Throughput Method for the Simultaneous Determination of 25-Hydroxyvitamin D3, Metanephrine, Normetanephrine and 3-Methoxytyramine in Plasma

### Materials

HPLC-grade methanol was obtained from Rathburn Chemicals Ltd. (Walkerburn, Scotland, UK); formic acid (ULC-MS quality) was obtained from Biosolve (Valkenswaard, The Netherlands); ammonium acetate, ascorbic acid, and di-Sodium hydrogen phosphate dihydrate were acquired from Merck KGaA (Darmstadt, Germany. 25-hydroxyvitamin D3 (25 OHVD3) was purchased from Sigma Aldrich (St. Louis, Mo.). A certified 25-hydroxyvitamin D3 reference standard from the Nation Institute of Standards and Technology was used to correct for impurity of the Sigma material (correction factor). Deuterated d6-25 OHVD3 was purchased from Medical Isotopes (USA) for use as an internal standard. We purchased D,L-metanephrine-HCl, D,L-normetanephrine-HCl, and D,L-3-methoxytyramine-HCl from Sigma-Aldrich, the deuterated internal standards α,α,β-d3-metanephrine-HCl and α,α,β,β-d4-3-methoxytyramine-HCl from Cambridge Isotopes, and α,α,β-d3-normetanephrine-HCl from Medical Isotopes; Reagent-grade water, obtained from a Barnstead system, was used throughout the study procedure.

A mix working solution of 25 OHVD3, metanephrine, normetanephrine and 3-methoxytyramine was diluted with ascorbic acid (400 mg/L in 10% methanol final dilution) from separate stock solutions in methanol on the day of analysis. Stock solutions were stored at −20° C. for a maximum of 6 months. The deuterated internal standard stock and mix working solution were treated the same way. Plasma calibrators were prepared by addition of the working solution mix in the approx. concentration ranges of 0-20 nmol/L for metanephrine, 0-203 nmol/L for normetanephrine and 0-20 nmol/L for 3-methoxytyramine and 0-200 nmol/L for 25-OHVD3. All the solutions were prepared in blank plasma. Final working internal standard solution was a mix of all 4 compounds created by serial dilution to the following concentrations: 25OHVD3=˜0.4 μmol/L, metanephrine=˜1.0 nmol/L, normetanephrine=˜1.5 nmol/L, 3-methoxytyramine=˜1.0 nmol/L in 50% acetonitrile. Blank plasma was obtained by treating plasma with pepsin and dialyzing this afterwards for three days against phosphate buffered saline.

Pepsin (≧2,500 units/mg protein) was purchased from Sigma Aldrich (St. Louis, Mo.). A working solution of 425 mg pepsin in 100 mL 1M hydrochloric acid was freshly prepared and stored at 4° C. until use. Vitamin C/EDTA solution was prepared by dissolving 100 mg vitamin C and 400 mg EDTA in 250 mL reagent-grade water. Phosphate buffer was prepared by dissolving 11.1 gram di-sodium hydrogen phosphate and 404 mg of EDTA in 500 mL of reagent-grade water.

### Sample Preparation:

Pipette 100 μL sample into a 96-deep-well plate. Add 50 μL of internal standard working solution to all samples and calibrators, add 300 μL of phosphate buffer and mix for 1 min on a plate shaker (600 rpm). Metanephrine, normetanephrine and 3-methoxytyramine were derivatized according to Manz et al. (GIT Labor-Medizin 1990; 5:245-253). After 15 min incubation on a shaker, 50 μL of pepsin solution was added and further incubated for 1 hour on a shaker at room temperature.
Add 50 μL of 1M sodium hydroxide and fill up the volume to 1.0 mL with reagent-grade water. Place the 96-deep-well-plate into the autosampler.

### Online Extraction and LC-MS/MS:

Online extraction and HPLC chromatography of the standards and samples was performed using a Spark Holland Symbiosis® online SPE system. See Example 1 for details of this system.
The cartridges (dimension 2×10 mm) used for the SPE were packed with a mix of four different immunoaffinity sorbents containing immobilized antibodies raised against, respectively, 25-OHVD3, metanephrine, normetanephrine and 3-methoxytyramine. The antibodies were prepared based on published methods. Antibody against 25-OHVD3 was prepared according to Kobayashi et al. (J. Steroid Biochem. Molec. Biol. 1994; 48: 567-572). Antibody against metanephrine, normetanephrine and 3-methoxytyramine was described in Manz et al. (GIT Labor-Medizin 1990; 5: 245-53).

The analytical column is an XSelect CSH Fluoro-Phenyl 100×2.0 mm, 2.5 μm from Waters. 100 μL of the standards or samples was injected onto the immunoaffinity cartridge. The online immunoaffinity procedure is performed as shown in example 1. The LC settings employed: 10 mm ammonium acetate, 0.1% formic acid in 10% methanol in water (eluent A) and 0.1% formic acid in methanol (eluent B). Gradient elution was performed according to the following elution program: 0 min, 80% A, 20% B; 6 min, 0% A, 100% B; 6-7.5 min, 0% A, 100% B; reequilibration from 7.5 to 9 min with 80% A, 20% B. Gradients applied were linear; flow rate was 0.300 mL/min. Column temperature was kept at 25° C. MS/MS settings from example 1 were applied.

### Example 6

### Kit for Detecting Testosterone

An exemplary kit of the invention comprises the following components:
Analytical column, preferably C18 bonded phase such as HALO C18 2.1×100 mm, 2.7 μm from Advanced Materials Technology,
Immunoaffinity cartridges, packed with resin containing immobilized antibodies against testosterone, 10×2.0 mm cartridges (96 in plate)
1.5 mL glass vials
Lyophilized pepsin
Internal standard, containing lyophilized deuterated d₃-testosterone stock
Calibrator, containing lyophilized testosterone stock
1.0M sodium hydroxide
SPE buffer 1: 0.1% formic acid in water
SPE buffer 2: 25 mM ammonium acetate, pH 7.2
SPE buffer 3: water
SPE buffer 4: 0.1% FA in 10% acetonitrile
SPE buffer 5: 0.1% FA in 20% acetonitrile
Mobile phase A: 2 mM ammonium acetate in water+0.1% formic acid
Mobile phase B: 0.1% formic acid in acetonitrile/water (95/5)

### Example 7

### Kit for Multi-Analyte Detection

An exemplary kit of the invention comprises the following components:
Analytical column, preferably Fluoro-Phenyl bonded phase such as XSelect CSH Phenyl-Hexyl 2.1×100 mm, 2.5 μm from Waters,
Immunoaffinity cartridges, packed with resin containing immobilized antibodies against 25-OHVD3, metanephrine, normetanephrine and 3-methoxytyramine, 10×2.0 mm cartridges (96 in plate)
96-Well plate
Lyophilized pepsin
Internal standard, containing lyophilized deuterated d₆-25-OH-Vitamin-D3, d₃.metanephine, d₃-normetanephrine, d₄-3-methoxytyramine
Calibrator, containing lyophilized 25-OH-Vitamin-D3, metanephine, normetanephrine, 3-methoxytyramine stock
Blank plasma
Vitamin C/EDTA solution
Phosphate buffer
Derivatization reagent
Sodium hydroxide
SPE buffer 1: 0.1% formic acid in water
SPE buffer 2: 25 mM ammonium acetate, pH 7.2
SPE buffer 3: water
SPE buffer 4: 0.1% FA in 10% acetonitrile
SPE buffer 5: 0.1% FA in 20% acetonitrile
Mobile phase A: 10 mm ammonium acetate, 0.1% formic acid in 10% methanol in water
Mobile phase B: 0.1% formic acid in methanol
   (iv) optionally, buffers necessary for performing the online solid phase extraction
   (v) optionally, an analytical column, preferably a C18 column.

1. An automated high-throughput method for quantitating at least one low molecular weight protein-bound biomarker directly in an isolated biological sample, comprising the steps of:
(a) automated prepurification of the isolated biological sample using an effective amount of an acid protease having a pH optimum in the range of pH 1.0-5.0 under conditions that allow for digestion of one or more binding proteins until they can no longer bind the at least biomarker of interest;
(b) applying said protease-digested sample onto an on-line immunoaffinity solid phase extraction (SPE) column to capture at least part of said biomarker, wherein the immunoaffinity SPE column comprises an immunoaffinity sorbent comprising an antibody capable of binding the at least one biomarker of interest; and wherein the protease-digested sample is applied to the immunoaffinity SPE column without prior protein precipitation and/or centrifugation, followed by sequential washing of the solid phase;
(c) eluting from the immunoaffinity SPE column a fraction comprising said at least one biomarker directly onto a liquid chromatography (LC) column comprising an apolar stationary phase and subjecting it to LC-MS or LC-MS-MS measurements to determine the amount of at least one biomarker; and
(d) quantitating the biomarker(s) in the sample by correlation with standard samples.

2. The method according to claim 1, wherein said acid protease is pepsin.

3. The method according to claim 1, wherein the protease-digested sample is applied to the immunoaffinity SPE column without prior protein precipitation and centrifugation.

4. The method according to claim 1, wherein the biomarker is a steroid, sterol or indole, preferably wherein the biomarker is selected from the group consisting of glucocorticoids, mineralocorticoids, androgens, estrogens and progestagens.

5. The method according to claim 4, wherein said biomarker is selected from the group consisting of melatonin, vitamin D, testosterone, cortisol and precursors and metabolites thereof.

6. The method according to claim 1, wherein step (a) is followed by neutralizing said protease-treated sample containing acid protease to a pH value at which the acid protease is substantially inactive, preferably wherein the neutralization does not increase the volume of the sample to more than 110%.

7. The method according to claim 1 for the simultaneous detection of at least a first and a second low molecular weight biomarker of interest in a single sample, preferably a plasma sample, wherein at least one of the biomarker is a protein-bound biomarker, wherein the SPE column comprises an immunoaffinity sorbent comprising at least a first antibody capable of binding the first biomarker and a second antibody capable of binding the second biomarker.

8. The method according to claim 7, wherein the first biomarker is a steroid hormone, preferably testosterone, and wherein the second biomarker is a catecholamine.

9. The method according to claim 1, wherein the SPE column comprises an immunoaffinity sorbent comprising an antibody having a high affinity for the biomarker but which is capable of releasing the biomarker under uniform conditions.

10. The method according to claim 1, wherein the sorbent in the SPE column comprises a binding agent, preferably an antibody, capable of binding at least one sample component which interferes with quantitating said at least one biomarker, preferably wherein said interfering component is an interfering human plasma protein, such as rheumatoid factor.

11. The method according to claim 1, wherein the biological sample is obtained from a mammalian subject, preferably a human subject or a test animal.

12. The method according to claim 1, wherein said sample has a volume up to 50 microliter, preferably up to 35 microliter.

13. The method according to claim 1, wherein the biological sample is blood plasma, blood serum or saliva, preferably a blood plasma sample.

14. A test kit for quantitating a protein-bound biomarker directly in a biological sample, comprising
(i) an acid protease stock solution, preferably a pepsin stock solution;
(ii) an solid phase extraction (SPE) column comprising a sorbent capable of capturing the biomarker;
(iii) a labelled analog of said biomarker;
(iv) optionally one or more selected from the group consisting of an analytical column, buffers necessary for performing the online solid phase extraction and subsequent chromatographic separation and instructions for use in an automated high-throughput method using (LC-(MS/)MS).

15. The test kit according to claim 14 for detecting one or more vitamin D derivative(s), comprising
(i) pepsin,
(ii) an SPE column comprising spherical particles conjugated to an antibody against said vitamin derivative(s),
(iii) stock solutions of one or more of 25-hydroxyvitamin D3, 25-hydroxyvitamin D2, 1α,25-dihydroxyvitamin D2 and/or 1α,25-dihydroxyvitamin D3, as unlabelled and as deuterated version, preferably wherein the deuterated vitamins D are deuterated in positions 26 and 27;
(iv) optionally, buffers necessary for performing the online solid phase extraction
(v) optionally, an analytical column, preferably comprising a C18 bonded phase endcapped with a polar group.

16. The test kit according to claim 14 for detecting testosterone, comprising
(i) pepsin,
(ii) an SPE column comprising spherical particles with immobilized antibody directed against testosterone
(iii) stock solution of testosterone, as unlabelled and as deuterated version
(iv) optionally, buffers necessary for performing the online solid phase extraction
(v) optionally, an analytical column, preferably a C18 column.

17. The test kit according to claim 14 for detecting melatonin, comprising
(i) pepsin,
(ii) an SPE column comprising spherical particles immobilized with antibody directed against melatonin
(iii) stock solution of melatonin, as unlabelled and as deuterated version
(iv) optionally, buffers necessary for performing the online solid phase extraction
(v) optionally, an analytical column, preferably a C18 column.
